(19) United States
(12) Patent Application Publication
(10) Pub. No.: US 2002/0009452 A1
(43) Pub. Date: Jan. 24, 2002

(54) METHOD FOR ALTERING UNDESIRABLE IMMUNE RESPONSES TO POLYPEPTIDES

(76) Inventor: MICHAEL CAPLAN, WOODBRIDGE, CT (US)

Correspondence Address: Brenda Herschbach Jarrell Choate Hall & Stewart, Exchange Place, 53 State Street, Boston, MA, 02109, US

(21) Appl. No.: 09/247,406

(22) Filed: Feb. 10, 1999

(51) Int. Cl. G01N 33/53; G01N 33/567; G01N 33/574; C07H 21/04; C12P 21/04; A61K 39/395; C12N 9/02; C12N 7/01

(52) U.S. Cl. 424/171.1; 424/141.1; 424/130.1; 435/7.1; 435/7.2; 435/7.23; 435/69.7; 435/320.1; 435/235.1; 435/189; 435/252.3; 536/23.1; 536/23.4; 536/25.32

(57) ABSTRACT

Disclosed is a method for altering undesirable immune responses by identifying mutant polypeptides that exhibit less of an undesirable immune response while retaining one or more desired characteristics. Such polypeptides are safer and can be more efficacious when introduced into a human, other mammal, or other animal. Either the altered immune response or a surrogate for the immune response, referred to as a measurable immune characteristic, can be assessed in the method. Generally, the measurable immune characteristic can itself be an undesirable immune response, the measurable immune characteristic can be involved in an undesirable immune response, and/or an undesirable immune response can be mediated by the measurable immune characteristic. The disclosed method involves providing a collection of mutant polypeptides where the amino acid sequence of each mutant polypeptide differs in at least one position from a polypeptide of interest, identifying mutant polypeptides that exhibit less of the immune response than the polypeptide of interest, and identifying mutant polypeptides with less potential for eliciting an undesirable immune response that still retain the desired characteristic(s). The collection of mutant polypeptides can be provided by mutagenizing nucleic acid encoding a polypeptide of interest and expressing the mutagenized nucleic acid to produce mutant polypeptides. The method is especially useful for reducing immune responses involving both linear and/or conformational epitopes on a polypeptide of interest. This is possible because the recombinant polypeptides used as substrates for mutagenesis in the method are essentially full length, remaining structurally similar to the polypeptide of interest and displaying essentially the same conformational epitopes.

### BACKGROUND OF THE INVENTION

The disclosed invention is generally in the fields of protein modification and reduced immune responses, and specifically in the area of production of proteins and peptides that elicit less of an undesirable immune response.

While the immune system is elegant and highly efficient it can also react in inappropriate and undesirable ways or fail to react in appropriate or desirable ways, especially to proteins and peptides. A well-known example of undesirable immune response is the allergic reaction, where an individual exposed to a protein releases histamines and other mediators of inflammation. Additionally, proteins administered as therapeutics are often attacked and eliminated by an immune response before the full therapeutic result is achieved.

The development of novel protein-based strategies in the treatment of allergy and other diseases has been hampered by the requirement to avoid initiating or unleashing a significant patient immune response (DeMatteo et al., Transplantation 63:315-319 (1997)). For example, Wadhwa et al., Clin and Exp. Immunol. 104:351-358 (1996), reporting diminished effectiveness of GM-CSF due to immune response. Rosenchein et al., Israel J. Med. Sci. 27:541-545 (1991)), reported an immune response to pharmaceutical streptokinase while Lantin et al., Clin. and Exp. Rheumatology 12:429-433 (1994), and Zilliox et al., J. Clin. Immunol. 13:415-423 (1993), reported more serious immune responses to the same substances. Similarly, the use of viruses, viral vectors, or viral capsids for delivery of nucleic acids and other therapeutics is hampered by immune responses to the viral proteins (DeMatteo et al., Transplantation 63:315-319 (1997)).

Some attempts have been made to reduce immune reactions to a few well studied proteins. In one study, Ferreira et al., FASEB Journal 12:231-242 (1998), describes mutated birch allergens having lower IgE reactivity. Ferreira et al. empirically identified amino acids in the antigen to be mutated using an algorithm that identified amino acids putatively linked to functional characteristics of a family of proteins. The method of Ferreira et al. depended on the identification and comparison of different forms of the allergens, including allergens previously identified as being less allergenic. Although this identification process resulted in less reactive allergens, the selection of mutant sites was limited to those identified in the algorithm.

Collen and co-workers described a method of epitope mapping in proteins involving phage-displayed randomized staphylokinase variants passed over a column loaded with an anti-staphylokinase murine monoclonal antibody or anti-staphylokinase polyclonal human antibodies to identify variants that failed to react with antibodies on the column (Jespers et al., J. Molecular Biology 5:704-718 (1997)). This technique was limited to assessment of reactivity of the staphylokinase variants to single, non-human monoclonal antibody or to a polyclonal pool. This prevents efficient assessment of the full range of relevant antibody interactions to staphylokinase.

It would be desirable to develop an efficient and reliable technique through which proteins or peptides can be modified to preserve or increase their useful properties while minimizing or eliminating their potential for eliciting an undesirable immune response.

It is therefore an object of the present invention to provide a method for reducing undesirable immune responses to proteins and peptides.

It is another object of the present invention to provide proteins and peptides that elicit reduced immune responses.

It is another object of the present invention to provide a method for decreasing a measurable immune characteristic of proteins and peptides that is associated with an undesirable immune response.

It is another object of the present invention to provide proteins and peptides having a reduced measurable immune characteristic.

It is another object of the present invention to provide a method of producing safer and more efficacious proteins and peptides for introduction into animals.

### BRIEF SUMMARY OF THE INVENTION

Disclosed is a method for reducing or preventing undesirable immune responses by generating and/or identifying mutant polypeptides that fail to elicit, or elicit less of, an undesirable immune response while retaining one or more desired characteristics. Such polypeptides are safer and can be more efficacious when introduced into humans or other animals. The disclosed method involves providing a collection of mutant polypeptides where the amino acid sequence of each mutant polypeptide differs in at least one position from a polypeptide of interest, identifying mutant polypeptides that exhibit less of the immune response than the polypeptide of interest, and identifying mutant polypeptides with less potential for eliciting an undesirable immune response that still retain the desired characteristic(s). The collection of mutant polypeptides can be provided by mutagenizing nucleic acid encoding a polypeptide of interest and expressing the mutagenized nucleic acid to produce mutant polypeptides. Either the immune response itself or a surrogate for the immune response, referred to as a measurable immune characteristic, can be assessed in the method. Generally, the measurable immune characteristic can itself be an undesirable immune response, the measurable immune characteristic can be involved in an undesirable immune response, the measurable immune characteristic can be associated with an undesirable immune response, and/or an undesirable immune response can be mediated by the measurable immune characteristic.

The undesirable immune response to be reduced can be of any type and will generally be determined by the immune response that is known to be, or which is discovered to be, associated with the particular polypeptide of interest. The undesirable immune response can be either the presence or absence of an immune response. Thus, a reduction in an undesirable immune response can be either a reduction or increase in the underlying immune response. Potential undesirable immune responses include, for example, humoral immune responses, cellular immune responses, allergic responses, any causes or effects of these immune responses, production of neutralizing antibodies, reactivity to IgA antibodies, reactivity to IgD antibodies, reactivity to IgE antibodies, reactivity to IgG antibodies, reactivity to IgM antibodies, B cell activation, T cell activation, NK cell activation, or any combination of these or other immune reactions and interactions. Other potential undesirable immune responses include, for example, a lack of a humoral immune response, a lack of a cellular immune response, a lack of an allergic response, a lack of any cause or effect of these immune responses, a lack of production of neutralizing antibodies, a lack of reactivity to IgA antibodies, a lack of reactivity to IgD antibodies, a lack of reactivity to IgE antibodies, a lack of reactivity to IgG antibodies, a lack of reactivity to IgM antibodies, a lack of B cell activation, a lack of T cell activation, a lack of NK cell activation, or a lack of any combination of these or other immune reactions and interactions.

Where the undesirable immune response is an allergic reaction to a polypeptide such as a food allergen, the mutant polypeptides can be tested for a reduction in IgE reactivity of the polypeptides while retaining one or more desired characteristics of the polypeptide of interest. In this case, IgE reactivity can be considered the measurable immune characteristic. Where the mutant polypeptides are to be used in immunotherapy for allergic disorders, desired characteristics to be retained by the mutant polypeptides can include the ability of the polypeptide to mediate T cell activation and participate in any other immune response involved in allergic desensitization.

Where the undesirable immune response is neutralization of a therapeutic polypeptide such as streptokinase or GM-CSF, the mutant polypeptides can be tested for a reduction in IgG binding or T cell activation (and/or other aspects or mediators of general immune responses) by the polypeptides while retaining one or more desired characteristics of the polypeptide of interest. In this case, IgG binding or T cell activation can be considered the measurable immune characteristic. Where the polypeptide of interest is streptokinase, the desired characteristic to be retained by the mutant polypeptides should include the clot dissolving ability of streptokinase. Where the polypeptide of interest is GM-CSF, the measurable immune characteristic can be IgG binding and the desired characteristic should be the trophic activity of GM-CSF.

Where the undesirable immune response is neutralization or clearing of a therapeutic virus, viral vector, or viral capsid such as a retroviral vector, viral coat proteins can be used as the polypeptide of interest. The mutant polypeptides (mutant forms of the viral protein) can be tested for a reduction in T cell activation (and/or other aspects or mediators of general immune responses) by the polypeptides while retaining one or more desired characteristics of the polypeptide of interest. In this case, T cell activation can be considered the measurable immune characteristic. Where the polypeptide of interest is a viral protein present in a virus-based therapeutic, the desired characteristic to be retained by the mutant polypeptides should include viral or capsid assembly and mediation of infectivity or cell entry.

Reactions of the immune system to proteins are based on its detection of relevant linear and/or conformational epitopes. Most prior methods of identifying epitopes or altering immune responses to proteins involve testing of individual peptide fragments of the protein of interest. Generally, such peptides do not embody conformational epitopes that may be present in the protein and thus such conformational epitopes are not assessed by these techniques. While such prior methods are useful for identifying linear epitopes, the disclosed method is especially useful for reducing immune responses involving both linear and/or conformational epitopes on a polypeptide of interest. This is possible because the recombinant polypeptides used as substrates for mutagenesis in the method are essentially full length, remaining structurally similar to the polypeptide of interest and displaying essentially the same conformational epitopes.

The disclosed method is especially useful for designing safer and more efficacious forms of polypeptides that are, or are intended to be, introduced into humans, other mammals, or other animals. In particular, safer forms of polypeptides used as or in therapeutics or as food can be made with the disclosed method.

### DETAILED DESCRIPTION OF THE INVENTION

### Definitions

Allergen. An allergen is an antigen which elicits IgE production in addition to other isotypes of antibodies.

Allergic reaction. An allergic reaction is an immune response that is IgE mediated with clinical symptoms primarily involving the cutaneous (uticaria, angiodema, pruritus), respiratory (wheezing, coughing, laryngeal edema, rhinorrhea, watery/itching eyes), gastrointestinal (vomiting, abdominal pain, diarrhea), and cardiovascular (if a systemic reaction occurs) systems.

Animal. As used herein, animal refers to all multicellular members of the kingdom Animalia. All animals other than humans are referred to as non-human animals.

Antibody reactivity. Antibody reactivity refers to specific binding between an immunoglobulin and a polypeptide. This specific binding refers to the type of antigen-specific binding that is the hallmark of immunoglobulin interactions.

Antigen. An antigen is a molecule that elicits an immune response such as production of antibody (a humoral response) or an antigen-specific reaction with T cells (a cellular response).

Bioactivity. A bioactivity can be any biological effect or function that a polypeptide may have. For example, bioactivities include specific binding to biomolecules (for example, receptor ligands), enzymatic activity, hormonal activity, cytokine activity, and inhibition of biological activity or interactions of other biomolecules (for example, agonists and antagonists of receptor binding).

Cell. The terms cell, cell line, and cell culture are used interchangeably and all such designations include progeny cells.

Encode. A nucleic acid referred to as encoding a protein, peptide, polypeptide, or amino acid sequence means that the sequence of nucleotide residues in the nucleic acid corresponds to codons specifying the amino acids of the protein, peptide, polypeptide, or amino acid sequence.

Epitope. An epitope is a binding site having an amino acid motif of between approximately six and fifteen amino acids, which can be either bound by an immunoglobulin or recognized by a T cell receptor when presented by an antigen presenting cell in conjunction with the major histocompatibility complex (MHC). A linear epitope is one where the amino acids are recognized in the context of a simple linear sequence. A conformational epitope is one where the amino acids are recognized in the context of a particular three dimensional structure.

Expression, expressing. Expression refers to any or all of the steps giving effect to encoded genetic information, including transcription, RNA processing, translation, post-translational processing, transport, and polypeptide activity or function. Thus, for example, expressing a nucleic acid to produce a polypeptide refers to transcription of the nucleic acid and translation of the transcript to form the polypeptide.

Expression sequence. Expression sequence refers to nucleic acid sequences necessary for the expression of an operably linked coding sequence in a particular host organism. A nucleic acid is operably linked to another nucleic acid when it is placed into a functional relationship with the other nucleic acid.

Immune characteristic. An immune characteristic is any characteristic of a molecule that involves, interacts with, is responsive to, or is associated with an immune response or immune system molecule or cell. Particularly useful immune characteristics are those that can be measured (measurable immune characteristics) and those that are correlated and/or associated with an immune response. Immune responses can be a form of immune characteristic.

Immune response. An immune response is any effect in an animal body mediated by or involving the immune system. Immune responses include the direct reactions and responses of immune system molecules and cells as well as any indirect reactions and effects that result from stimulation of the immune system. Immune responses include any antibody reactivity, immune response—such as humoral immune responses, cellular immune responses, and allergic responses—T cell, B cell, and NK cell activation, and altered function of other immune system cells such as macrophages, mast cells, basophils, eosinophils, and dendritic cells(see, for example, Abbas et al., Cellular and Molecular Immunology (W. B. Saunders Co., Philadelphia, 1994)). Immune responses also include effects of immune system stimulation such as the effects of an allergic reaction where histamines and other mediators of inflammation are released or produced.

Immunomodulatory. An immunomodulatory molecule or polypeptide is a molecule that alters a feature or state of the immune system or immune system cells. Examples of immunostimulatory molecules include adjuvants and interleukins.

Mammals. Mammal refers to all types of mammals including humans, domesticated animals such as cows, sheep, goats, and horses, and pets such as cats and dogs. All mammals other than humans are referred to as non-human mammals.

Measurable immune characteristic. A measurable immune characteristic is an immune characteristic that can be measured in an assay or otherwise assessed. Measurable immune characteristics of polypeptides are what is assessed in the disclosed method. Generally, the measurable immune characteristic will be a characteristic associated with an undesirable immune response such that an alteration in the measurable immune characteristic will be indicative or predictive of an alteration in the immune response. Measurable immune characteristics associated with an undesirable immune response can be used as indicators or stand-ins for the determination of one or more components of an actual immune response in an animal.

Monospecific antibody. A monospecific antibody (or antibody fragment) is an antibody that recognizes a single epitope.

Mutagenized. Mutagenized nucleic acid and mutated nucleic acid refers to nucleic acid that has been made or processed to contain changes in nucleotide sequence (referred to herein as mutations). Mutagenizing nucleic acid refers to the process of producing mutagenized nucleic acid. Randomly mutagenized nucleic acids refers to a group of nucleic acids that have been mutagenized such that the group of nucleic acids collectively include a variety of mutations more or less randomly distributed both in type and in location within the nucleic acid.

Mutant polypeptide. Mutant polypeptide refers to a form of a polypeptide of interest that has an altered amino acid sequence relative to the polypeptide of interest.

Nucleic acid. Nucleic acid refers to a polynucleotide molecule or segment of a polynucleotide molecule. Preferably, nucleic acid for use in the disclosed method will be composed of RNA or DNA.

Peptide, polypeptide, protein. Protein refers to an amino acid chain of greater than 100 amino acids or a multimeric association of such amino acid chains. A peptide refers to an amino acid chain of from 5 to 99 amino acids. Peptides of any sub-range of 5 to 99 amino acids are specifically and separately contemplated. Preferred peptides for use with the disclosed method are peptides that include an epitope. As used herein, polypeptide refers to a protein or peptide. A polypeptide having a conformational epitope refers to proteins and peptides having a conformational epitope. Randomly mutant polypeptides refers to mutant polypeptides that are encoded by a group of randomly mutagenized nucleic acids.

Polypeptide of interest. As used herein, polypeptide of interest refers to the original polypeptide that is the focus of the disclosed method. The polypeptide of interest is the non-mutant form of polypeptide of which mutant forms are produced in the disclosed method. Use of the term “non-mutant” does not mean that the non-mutant polypeptide is non-mutant in any absolute sense. Rather, the term is used merely to reflect the relationship between the original polypeptide of interest and the mutant polypeptides produced in the disclosed method. Thus, for example, a “mutant” form of a polypeptide can be used as the polypeptide of interest and, in this context, becomes the “non-mutant” polypeptide.

Undesirable immune response. An undesirable immune response is any immune response that is subjectively undesirable. Thus, an immune response undesirable in one context may be desirable in another context.

### Description

To reduce or eliminate undesirable immune responses to polypeptides of interest, the disclosed method makes use of a pool of mutant forms of the polypeptide of interest from which forms having less potential for eliciting an undesirable immune response are identified. The disclosed method does not require any knowledge of the specific epitopes on the polypeptide of interest that mediate the undesired immune response. The disclosed method also has the advantage that polypeptides having less potential for eliciting an undesirable immune response can be identified even if multiple epitopes on the polypeptide are involved in causing undesirable immune responses. This is possible since most embodiments of the disclosed method involve testing the mutant polypeptides against immunoglobulins or their derivatives which are specific for individual epitopes. If multiple mutations are needed to abolish or reduce an undesirable immune response, they can be identified individually using the disclosed method and then combined in a single polypeptide including multiple mutations.

The undesirable immune response to be reduced or eliminated in the disclosed method is intended to include any immune response that is subjectively or objectively undesirable. For example, an immune response that neutralizes or clears virus introduced into a mammal can be desirable in the case of a viral infection but undesirable in the case of a virus-based therapeutic. For the disclosed method, any immune response can be viewed as an undesirable immune response. Generally, all that is required is a subjective belief that the target immune response is undesirable.

Undesirable immune responses can take many forms. For example, it is not uncommon for proteins and peptides introduced into patients to stimulate immune responses. An immune response that serves to eliminate foreign proteins from the body can drastically reduce the effectiveness of a therapy by clearing the therapeutic protein from the body or preventing it from having its desired effect. An example of this is GM-CSF, where antibodies which neutralize GM-CSF function occur in approximately 40% of patients (Wadhwa et al., Clin and Exp. Immunol. 104:351-358 (1996)). Similarly, Rosenchein et al., Israel J. Med. Sci. 27:541-545 (1991)), reported non-threatening immune response to streptokinase while Lantin et al., Clin. and Exp. Rheumatology 12:429-433 (1994), and Zilliox et al., J. Clin. Immunol. 13:415-423 (1993), reported more serious immune responses. Undesirable immune response to viruses recruited for therapeutic purposes have also been described (DeMatteo et al., Transplantation 63:315-319 (1997)). Other polypeptides may induce IgA, IgD, IgE, IgG, or IgM responses, or may stimulate cellular rather than humoral immune responses. Introduced proteins may also produce an objectively undesirable immune response such as an allergic reaction.

Some immune responses are mediated by immunoglobulins and the disclosed method can make use of reactions between polypeptides and such immunoglobulins to assess when the potential for an immune response by a mutant polypeptide is reduced. For this purpose, the particular type(s) of immunoglobulins involved in the immune response (or their derivatives) should be used in the disclosed method. For example, IgE is known to mediate allergic responses. Other immune responses are mediated by immune system cells. Many techniques for assessing immune characteristics and immune responses have been developed and can be used in the disclosed method for identifying polypeptides having less potential for eliciting an undesirable immune response. Many techniques are also known for manipulating immunoglobulins and immune system cells for use in such immune assays, including the production of immunoglobulin libraries, recombinant immunoglobulins, and immunoglobulin fragments.

The disclosed method is most efficiently performed by using only immunoglobulins (or corresponding immunoglobulin fragments or immune system cells) that are reactive with the polypeptide of interest. In this way only relevant immune interactions are assessed. It is most preferred that only immunoglobulins (or derivatives) that are known or suspected to be involved in the undesirable immune response to the polypeptide be used. An effective means of achieving these goals is to use immunoglobulins derived from patients known to exhibit the undesirable immune response to the polypeptide of interest.

Immunoglobulins (and related derivatives) include IgA antibodies, IgD antibodies, IgE antibodies, IgG antibodies, and IgM antibodies. Immune system cells relevant to the disclosed method include B cells, T cells, NK cells, macrophages, mast cells, basophils, eosinophils, dendritic cells, and other immune system cells. The characteristics, functions, preparation, and use of such immunoglobulins and immune system cells are generally known (Abbas et al., Cellular and Molecular Immunology (W. B. Saunders Co., Philadelphia, 1994); Johnstone and Thorpe, “Immunochemistry in Practice,” Third Edition (Blackwell Scientific Publications, Oxford, 1996); Harlow and Lane, “Antibodies-A Laboratory Manual” (Cold Spring Harbor, 1988), “Monoclonal Antibodies” (Kennett et al., eds., Plenum Press, 1980); Steward and Steensgaard, “Antibody Affinity: Thermodynamic Aspects and Biological Significance” (CRC Press, 1983)). Examples of such techniques are described below.

Where the undesirable immune response is an allergic reaction to a polypeptide such as a food allergen, the mutant polypeptides can be tested for a reduction in IgE reactivity of the polypeptides while retaining one or more desired characteristics of the polypeptide of interest. In this case, IgE reactivity can be considered the measurable immune characteristic since IgE reactivity mediates allergic reactions. The mutant polypeptides are preferably tested against a set of IgE antibodies (or antibody fragments) reactive with the polypeptide of interest and derived from patients that are allergic to the polypeptide of interest. This makes it more likely that relevant epitopes will be mutated. Where the mutant polypeptides are to be used in immunotherapy, desired characteristics to be retained by the mutant polypeptides can include the ability of the polypeptide to mediate T cell activation and any other immune response involved in desensitzation.

Where the undesirable immune response is neutralization of a therapeutic polypeptide such as streptokinase or GM-CSF, the mutant polypeptides can be tested for a reduction in IgG binding or T cell activation (and/or other aspects or mediators of general immune responses) by the polypeptides while retaining one or more desired characteristics of the polypeptide of interest. Different immune responses may be stimulated by introduction of streptokinase or GM-CSF. In these cases, IgG binding or T cell activation can be considered the measurable immune characteristic. Where the polypeptide of interest is streptokinase, the desired characteristic to be retained by the mutant polypeptides should include the clot dissolving ability of streptokinase (that is, the activity that makes streptokinase usefull). Where the polypeptide of interest is GM-CSF, the desired characteristic to be retained by the mutant polypeptides should be the trophic activity of GM-CSF.

Where the undesirable immune response is neutralization or clearing of a therapeutic virus, viral vector, or viral capsid such as a retroviral vector, viral coat proteins can be used as polypeptides of interest. The mutant polypeptides (mutant forms of the viral protein) can be tested for a reduction in T cell activation, immunoglobulin binding, and/or other aspects or mediators of general immune responses, by the polypeptides while retaining one or more desired characteristics of the polypeptide of interest. In this case, the assessed T cell activation, immunoglobulin binding, or other aspect or mediator of general immune responses can be considered the measurable immune characteristic. Desired characteristics to be retained by the mutant polypeptides should include viral or capsid assembly and mediation of infectivity or cell entry. That is, the viral proteins should be able to assemble into infective particles that can mediate the therapeutic purpose. For this purpose, infectivity refers to cell entry and, if relevant, expression of viral genes, but not necessarily pathogenesis.

### Method

The disclosed method involves providing a collection of mutant polypeptides where the amino acid sequence of each mutant polypeptide differs in at least one position from a polypeptide of interest, identifying mutant polypeptides that exhibit less of an undesirable immune response than the polypeptide of interest and that still retain one or more desired characteristic(s). The collection of mutant polypeptides can be provided by mutagenizing nucleic acid encoding a polypeptide of interest and expressing the mutagenized nucleic acid to produce mutant polypeptides. The mutant polypeptides exhibiting less of an undesirable immune response can be identified by identifying an alteration in a measurable immune characteristic associated with the undesirable immune response. For example, the undesirable immune response can be mediated by the measurable immune characteristic assessed in the method, the assessed measurable immune characteristic can be involved in the undesirable immune response, or the assessed measurable immune characteristic can itself be the undesirable immune response. Such relationships are not mutually exclusive and multiple relationships between the measurable immune characteristic assessed and the undesirable immune response are contemplated and can be targeted in the disclosed method.

A. Mutation of Nucleic Acid

In the disclosed method, the nucleic acid encoding a polypeptide of interest can first be mutagenized to produce a set of randomly mutagenized nucleic acids collectively encoding a set of randomly mutant polypeptides. These mutant polypeptides are the raw material from which polypeptides that fail to elicit or that elicit less of an immune response are selected. Mutagenizing nucleic acid refers to the process of producing mutagenized nucleic acid. As used herein, mutagenizing is not limited to any particular method of creating the changes in the nucleic acid. For example, mutagenizing encompasses chemical synthesis of nucleic acids of pre-determined (albeit altered) sequence as well as low fidelity PCR and traditional methods of creating genetic mutations. Randomly mutagenized nucleic acids refers to a group of nucleic acids that have been mutagenized such that the group of nucleic acids collectively include a variety of mutations more or less randomly distributed both in type and in location within the nucleic acid. It is understood, however, that nucleic acids are still referred to as randomly mutagenized nucleic acids when the method of mutagenesis is such that only certain types of mutations are formed or certain types of mutations are favored over others. It is also understood that the process of producing such randomly mutagenized nucleic acids need not involve random or non-directed changes (although this may be preferred). All that is required is the result: a set of nucleic acids collectively including a variety of mutations more or less randomly distributed both in type and in location within the nucleic acid.

The nucleic acid can be mutagenized using any suitable technique. Many methods of mutagenesis are known and can be used with the disclosed method. Random mutagenesis methods, that is, methods that are not site-directed, are preferred. Preferred methods include low fidelity PCR amplification, chemical mutagenesis, or X-ray mutagenesis.

The mutagenesis step can be illustrated by the following example involving low fidelity PCR. When low fidelity PCR is used to perform random mutagenesis of nucleic acid encoding the polypeptide of interest, techniques such as those described by Parent and Devreotes (Parent and Devreotes, J. Biol. Chem. 270:22693-22696 (1995)) can be employed. Briefly, nucleic acid encoding the polypeptide of interest (such as a plasmid containing the cDNA encoding the polypeptide) can be used as the template in a PCR reaction. The primers can be chosen to amplify either the entire coding sequence or a portion of the coding sequence of the polypeptide of interest. The primers encode restriction sites that can facilitate subcloning of the PCR products directly into an expression vector (when the entire polypeptide sequence is to be amplified) or into the appropriate sites of the polypeptide sequence pre-inserted into an expression vector (when a portion of the antigen sequence is to be amplified). The latter allows mutagenesis directed to a portion of the polypeptide.

PCR can then be performed under conditions that render the polymerase prone to incorporation errors. Such conditions include, for example, the use of 5 units of Taq polymerase in the presence of 6 mM MgCl₂, 0.5 mM MnCl₂, 10 mM Tris HCl pH 8.3, 50 mM KCl, 0.01% gelatin, 0.5 mM dATP and 1 mM each of dCTP, dTTP and dGTP. The temperatures and durations of the steps of the PCR protocol can be optimized for each polypeptide sequence and primer set using known techniques. In general, 30 cycles of PCR should be performed. Following PCR amplification, the products can be cut with appropriate restriction enzymes and subcloned into an expression vector. The resultant library of expression plasmids containing randomly mutated nucleic acid encoding the polypeptide of interest can be transformed into bacteria (or other appropriate cells) and expressed.

Other examples of the PCR mutagenesis techniques are described by Parikh and Guengerich, Biotechniques 24(3):428-31 (1998); Loreno and Blasco, Biothechniques 24(2):308-13 (1998); Lin-Goerke et al., Biotechniques 23(3):409-12 (1997); Kawasaki et al., J. Biol. Chem. 272(25):15668-74 (1997); Burns et al., J. Biol. Chem. 271(27):15879-83 (1996); and Light and Lerner, Bioorgan. Medicinal Chem. 3(7):955-67 (1995). An example of random chemical mutagenesis is described by Encell et al., Cancer Res. 58(5):1013-20 (1998). Zaccolo et al., J. Mol. Biol. 255(4):589-603 (1996), describes a method of mutagenesis involving incorporation of nucleoside analogues during DNA synthesis. Little et al., J. Biotechnology 41:187-95 (1995), describes a method of random mutagenesis using random oligonucleotides. These methods and others can be used in the disclosed method.

It is preferred that the nucleic acid encoding the polypeptide of interest be mutagenized prior to insertion into the vector in which it will be expressed. This eliminates spurious mutations to the vector or expression sequences. This is not essential, however, since most spurious vector mutations will prevent expression of the polypeptide. Where the polypeptide of interest is to be fused to another polypeptide (such as an immunomodulatory polypeptide or a reporter protein), the nucleic acid encoding the polypeptide of interest can be mutagenized either separately or nucleic acid encoding the fusion protein can be mutagenized. Fusion of polypeptides refers to any coupling of two or more polypeptides, preferably via normal peptide bonds. It is preferred that the nucleic acid encoding the polypeptide of interest be mutagenized separately to avoid undesirable mutations in the other polypeptide.

B. Expression of Nucleic Acid

After mutagenesis, the mutagenized nucleic acid is expressed to produce the encoded mutant polypeptide. Expression can be accomplished using any suitable technique of gene expression. For example, suitable expression systems are available for bacterial cells, yeast cells, other fungal cells, insect cells, mammalian cells, and in vitro synthesis. Many such techniques are known and most can be used with the disclosed method. Some examples for the expression of mutant polypeptides are described in Methods in Enzymology, Vol. 153, Chapters 23 to 34 (Wu and Grossman, eds., Academic Press, 1987), and The 1995 Lab Manual Source Book (Cold Spring Harbor Laboratory Press, NY, 1995). The expression of the mutagenized nucleic acid is facilitated by known expression sequences, vectors, and cell strains. All that is required is expression of the mutant polypeptide such that an immune response or measurable immune characteristic of the mutant polypeptide can be measured or detected. In many cases, this does not require purification of the mutant polypeptide.

Although it is preferred that the mutant polypeptides used in the disclosed method be provided by mutagenizing nucleic acid encoding the polypeptide of interest followed by expression of the mutagenized nucleic acid, this is not required. The mutant polypeptides can be produced using any suitable method. For example, the mutant polypeptides can be made by direct chemical synthesis or chemical or enzymatic linkage of peptides.

C. Identifying Mutant Polypeptides That Elicit Less Of An Immune Response

After expression of the mutagenized nucleic acid, mutant polypeptides that elicit less of an immune response are identified. The potential of mutant polypeptides to elicit an undesirable immune response can be assessed using any suitable assay, including assays for measurable immune characteristics associated with the undesirable immune response. For example, where the measurable immune characteristic is reactivity to an antibody, less reactive polypeptides are selected by exposing the mutant polypeptides to individual antibodies or antibody fragments. The mutant polypeptides should, where possible, be tested against individual antibodies or antibody fragments since individual mutants would not be expected to have reduced reactivity to all antibodies reactive with the polypeptide of interest. It is preferred that the mutant polypeptides be tested only against antibodies or antibody fragments that are reactive to the polypeptide of interest. It is also preferred that the antibodies or antibody fragments be derived from patients or other animals exhibiting or predisposed to undesirable antibody reactivity to the polypeptide of interest.

The antibody or antibody derivative can be, for example, a recombinant Fab fragment, a monoclonal antibody prepared from mammalian hybridomas such as human or mouse, or a monospecific antibody fractionated from a polyclonal serum. Preparation of antibodies and antibody fragments, both individually and as part of a library, are described below. Any of these or other sources of antibodies can be used. Antibody reactivity can be measured using one of many suitable methods such as by immunoprecipitation reaction, radioimmune assays (RIA), immunoradiometric assay (IRMA), and enzyme-linked immunosorbent assays (ELISA). These and other useful methods for determining antibody reactivity are described in Johnstone and Thorpe, “Immunochemistry in Practice,” Third Edition (Blackwell Scientific Publications, Oxford, 1996).

The identification step in the disclosed method is illustrated by the following example involving a bacterial phage display library of mutagenized polypeptides. The library can be replica plated onto filters and expression of the mutagenized polypeptides can be induced (through incubation of the filters with IPTG, for example). Bacterial colonies can then be lysed and non-specific binding sites blocked through incubation with a solution such as phosphate buffered saline (PBS) containing 3% bovine serum albumin (BSA). Subsequently the filters can be incubated with antibody (or derivative) directed against the polypeptide of interest. After this incubation, unbound antibody can be removed by repeated washing.

Bound antibody can then be detected. For example, bound antibody can be detected by incubating with a secondary antibody coupled to a detectable reporter. This reporter can be an enzyme (for example, horseradish peroxidase, alkaline phosphatase) or a fluorochrome (such as fluorescein or rhodamine). In the case of recombinant Fabs, sequences encoding green fluorescent protein (GFP) can be appended to sequences encoding the Fabs themselves, thus fusing a detectable marker directly to the Fabs and obviating the need for incubation with a marker-tagged secondary antibody. Unbound secondary antibody can be removed by washing and bound secondary antibody can be detected by enzymatic assay or through measurements of fluorescence. Those clones that do not react with antibody are thus identified as producing mutant polypeptides that have less of the measurable immune characteristic (antibody reactivity) and less potential for eliciting an undesirable immune response. The corresponding colonies can be picked and expanded, and the DNA encoding the mutant polypeptides can be recovered and sequenced.

Techniques for detecting and measuring immune responses, T cell activation and B cell activation are also known and can be used to assess the potential to elicit an immune response in the disclosed method. For example, to determine whether a mutant polypeptide retains the ability to activate T cells, the following standard assay, which is described in Current Protocols in Immunology, volume 1, pages 3.12. lff (Coligan et al., eds., John Wiley and Sons), can be performed. The peripheral blood lymphocytes of humans or other mammals that manifest an immune response to the polypeptide of interest can be isolated from whole blood using ficoll histopaque. Individual recovered cells can be washed and suspended in media at the concentration of 4×10⁶ cells/ml. For the proliferation assays, 6 wells of a 96 well plate at 2×10⁵ PBLs/well can be stimulated in triplicates with media (control) or an appropriate quantity of polypeptide at 37° C. The cells in the 96 well plates should be allowed to proliferate in the absence (media) and presence of the polypeptide for 6 days. On day 6 the cells can be treated with radioactive thymidine (1 μCi/well), re-incubated at 37° C. for 6 to 8 hours, and harvested onto glass fiber filters. T cell proliferation can be estimated by quantitating the [³H]-thymidine incorporation into the DNA of proliferating cells. [³H]-thymidine incorporation is reported as stimulation (SI) above media treated (control) cells. Any polypeptide that induces an SI above 2.0 will be considered stimulatory.

Many other useful techniques for detection and determination of antibody reactivity and immune responses are described in Harlow and Lane, “Antibodies-A Laboratory Manual” (Cold Spring Harbor, 1988), “Monoclonal Antibodies” (Kennett et al., eds., Plenum Press, 1980), and Steward and Steensgaard, “Antibody Affinity: Thermodynamic Aspects and Biological Significance” (CRC Press, 1983).

In the case of known allergens, polypeptide-based pharmaceuticals already in use (for example, streptokinase), or polypeptide-based pharmaceuticals which have already been tested, monoclonal antibodies directed against the polypeptide can be derived as described elsewhere herein. Preferably, patients (or test subjects) who are already allergic to an allergen or have been exposed to the drug are used as the source for peripheral blood monocytes to be used in the production of recombinant immunoglobulin phage display libraries as described elsewhere herein. It is more preferable that such individuals who exhibit the undesirable immune response are used as the source. The type of immunoglobulin phage display library (that is, IgG, IgE, IgA, IgD, IgM) can be tailored to the characteristics of the immune response associated with the particular polypeptide. The relevant reactive monoclonal immunoglobulin species can be selected by panning with the polypeptide of interest (that is, the non-mutant polypeptide) and subsequently used to identify less reactive mutant polypeptides in the disclosed method.

In the case of novel polypeptide-based agents (or other polypeptides) which have yet to be used or tested, the polypeptide can be administered to an animal and its peripheral blood monocytes can be used in the production of the recombinant immunoglobulin phage display library. Alternatively, monoclonal antibodies directed against the polypeptide-based therapeutic agent can be generated directly by standard methods for the production of hybridomas. This approach will allow the polypeptide to be modified in a manner that will reduce its potential to elicit an immune response.

An alternative to using animal models for those polypeptides that have yet to be tested in humans involves the production of recombinant combinatorial phage display immunoglobulin libraries from “naïve”, individuals (that is, individuals who have not been exposed to the polypeptide). A representative repertoire of potential humoral responses is present in the B cells of non-immunized individuals, although B cells that have not undergone clonal expansion in response to an appropriate antigen are present at low multiplicity. Methods have been developed to produce representative “naïve” combinatorial libraries (Sodoyer et al., Human Antibodies 8:37-42 (1997)). Such methods can be used to produce recombinant Fab or other antibody fragments that react with the antibody of interest. These epitope-specific monoclonal recombinant antibodies can then be used in the disclosed method to screen for mutations that reduce or eliminate antibody binding. By mutagenizing to eliminate reactivity with any polypeptide-specific immunoglobulin that might be present in the complete B cell repertoire, it should be possible to reduce the potential of protein-based drugs that have not yet been used or tested to elicit an undesirable immune response. Once forms of the polypeptide that elicit less of an undesirable immune response have been identified, these polypeptides can be further tested for the introduction of different undesirable immune responses or other side effects.

It is preferred that, prior to determining the potential for eliciting an undesirable immune response, the expressed mutant polypeptides be screened to eliminate those that have gross mutations such as frameshifts and large deletions. This is preferably accomplished by expressing the mutant polypeptide as a fusion protein with a reporter protein. In most cases of gross mutation, the reporter protein will not be expressed or will not be functional. Thus, gross mutations can be screened out by looking for expression of the reporter protein and eliminating those mutant proteins where the reporter protein is not expressed.

The reporter sequences should be placed such that when the mutagenized nucleic acid encoding the polypeptide of interest is inserted, a fusion protein will be formed in which the detectable reporter is appended to the COOH terminus of the mutant polypeptide. This can be accomplished, for example, by designing the 3′ PCR primer used in the mutagenesis in such a way that the polypeptide's stop codon is removed and the polypeptide-encoding sequences are appended to the reporter-encoding sequences in the same reading frame. The detectable reporter can be, for example, an enzyme (such as horseradish peroxidase or alkaline phosphatase), a fluorochrome (such as green fluorescent protein) or an epitope tag that is recognized by a monospecific antibody. The set of mutagenized fusion nucleic acids can then be screened both for the potential for eliciting an undesirable immune response (as described above) and for expression of the COOH terminal reporter. Only those clones that express the reporter but exhibit a reduction in the measurable immune characteristic should be chosen for further study. By selecting only those clones, it is established that the mutations that eliminate or reduce the potential for eliciting an undesirable immune response are not trivially attributable to large deletions, premature terminations, frame shifts, or other gross mutations. If, for example, GFP is used for the COOH terminal reporter while a rhodamine-conjugated secondary antibody is used to report antibody binding to the antigen, only those colonies which produce a GFP but not a rhodamine fluorescent signal should be retained. By choosing appropriate secondary antibody and fusion protein reporters (for example, the use of two fluorescent labels) the detection steps in the disclosed method can be automated.

After screening as described above, the identified mutant polypeptides can be further tested by assessing whether the undesirable immune response is reduced or eliminated.

D. Combining Mutations

Once mutant polypeptides are identified that retain the desired characteristic(s) and elicit less of the measurable immune characteristic or the undesirable immune response, the mutations in individual mutant polypeptides can be identified. Once identified, the mutations can be combined in a single mutant polypeptide to produce a mutant polypeptide that elicits even less of the undesirable immune response. This is desirable since, for example, multiple antibodies may be responsible or involved in the undesirable immune response to a given polypeptide. Once multiple mutations are combined in one polypeptide, the polypeptide should be tested for retention of the desired characteristic(s).

The combination of different mutations in a single polypeptide can be accomplished using any suitable recombinant DNA techniques. Many such techniques are known and available. For example, one mutation can be combined with another mutation by site directed mutagenesis of nucleic acid encoding the first mutant polypeptide such that the nucleic acid sequence is altered to encode the specified second mutation.

Mutant polypeptides identified in the disclosed method are useful for introduction into an animal for any desired purpose, including as a therapeutic or as food. The reduced or eliminated measurable immune characteristic should reduce the risk of an undesirable immune response.

E. Identifying Mutant Polypeptides That Retain Desired Characteristics

The mutant polypeptides can also be screened to identify those that retain one or more desired characteristics. Preferred characteristics for retention include T cell activation, enzymatic activity, bioactivity, and the ability to promote allergic desensitization. The method used to detect such characteristics depends on the characteristic involved. In general, it is contemplated that desired characteristics will be a characteristics that are known for the polypeptide of interest and for which a method of detection is available. Some characteristics, such as antibody reactivity and T cell activation, can be measured using generalized techniques that do not depend on the specific polypeptide involved. Such methods are known and can be applied to any of the disclosed mutant polypeptides. If desired, the mutant polypeptides can be screened for retention of multiple desired characteristics. If the desired characteristic is T cell activation, such activation can be measured as described above. Ultimately, if not done as part of the disclosed method, the identified polypeptides can be tested in humans, other mammals, or other animals for both reduction in the undesirable immune response and retention of a desired effect.

The identification of mutant polypeptides having a reduced measurable immune characteristic and the identification of mutant polypeptides that retain one or more desired characteristics can be performed in any order or simultaneously. That is, identification of mutant polypeptides that exhibit less of, or have less potential to exhibit, the undesirable immune response can be carried out prior to, simultaneous with, or following identification of mutant polypeptides that retain the desired characteristic. Where one of the characteristics is screened first, it is preferred that only those mutant polypeptides identified in the first screen be subjected to the second identification. For example, where a set of mutant polypeptides exhibiting less of, or having less potential to exhibit, the undesirable immune response are first identified, only these identified polypeptides need be tested for retention of the desired characteristic.

F. Illustration

The following illustrates an example of how the disclosed method can be used in the context of allergens. Allergic disease is a common health problem affecting humans. Allergy is manifested by the release or production of histamines and other mediators of inflammation by mast cells or related cells which are triggered into action when IgE antibodies bound to receptors on the mast cell surface are cross-linked by antigen. Other than avoidance, and drugs (for example, antihistamines, decongestants, and steroids) that only can modify symptoms, may have unwanted side effects, and often only provide temporary relief, the only currently medically accepted treatment for allergies is immunotherapy.

Traditional immunotherapy involves the repeated injection of allergen extracts, over a period of years, to desensitize a patient to the allergen. Unfortunately, traditional immunotherapy can be dangerous and uncomfortable due to the allergic response of patients to the allergen extract itself. For some allergens immunotherapy is not possible because even low levels of allergen can produce life-threatening reactions in some patients, caused by, for example, mast cell degranulation leading to anaphylaxis. These problems can be ameliorated with the disclosed method.

In the context of the disclosed method, the undesirable immune response is the allergic response. Since IgE mediates allergic responses to allergens, the preferred measurable immune characteristic to be assessed in the case of an allergen is IgE reactivity. Where the goal is to produce a form of the allergen that is less allergenic but which can still be used for effective desensitization immunotherapy, the desired characteristic to be retained is a characteristic involved in desensitization. In the disclosed method, this characteristic can be detected by assessing T cell activation.

The method is best carried out using IgE antibodies or antibody fragments derived from patients that are known to be allergic to the allergen. Standard techniques can be utilized to generate a combinatorial antibody library from a patient allergic to an allergen (Steinberger et al., J. Biol. Chem. 271:10967-10982 (1996)). A combinatorial IgE phage display library from mRNA isolated from the peripheral blood mononuclear cells of one or more allergic patients can be prepared. Allergen-specific IgEs can be selected by panning filamentous phage expressing IgE Fabs on their surfaces against allergen immobilized on the walls of 96 well microtiter plates. The cDNAs can then be isolated from allergen-binding phage and transformed into E. coli for the production of large quantities of monoclonal, recombinant, allergen-specific IgE Fabs.

To determine whether an antibody library includes a complete inventory of the polypeptide-specific IgE antibodies present in patient serum, an immunocompetition assay can be performed. For this, pooled recombinant Fabs can be preincubated with immobilized polypeptide of interest. After washing to remove unbound Fab, the immobilized polypeptide would then be incubated with patient serum. After washing to remove unbound serum proteins, an incubation with a reporter-coupled secondary antibody specific for the antibody Fc domain can be performed. Detection of bound reporter would allow quantitation of the extent to which serum antibody was prevented from binding to polypeptide by recombinant Fab. Maximal, uncompeted serum antibody binding can be determined using polypeptide which had not been preincubated with Fab or had been incubated with nonsense Fab.

Mutant forms of a polypeptide allergen for testing can be produced by low fidelity PCR mutagenesis of nucleic acid encoding the allergen. The mutagenized nucleic acid can then be expressed to produce a pool of randomly mutant allergens for testing against the IgE library prepared as described above and for testing for T cell activation (the desirable characteristic). Since different epitopes on the allergen are expected to react with different allergen-specific IgEs, a mutant allergen that fails to react with even a single allergen-specific IgE should be carried forward in the method (the various different mutations involved can be combined later). For assessment of the desirable characteristic, mutant polypeptides can be tested for retention of reactivity with T cells. The two assays (for IgE reactivity and for T cell activation) can be performed in either order or simultaneously. Once allergens have been identified which both fail to react with an allergen-specific IgE and retain reactivity to T cells, the individual mutations can be identified. A composite allergen embodying mutations that abolish reactivity to different allergen-specific IgEs can then be made. This composite mutant allergen should then be tested against relevant allergen-specific IgEs and T cells to ensure that IgE reactivity is reduced while sufficient T cell reactivity is retained in the composite allergen. Finally, the composite mutant allergen can be used for immunotherapy with reduced risk of negative side effects.

### Materials

A. Antibodies

It is preferred that antibodies for use in the disclosed method be obtained and derived from humans or appropriate mammals, more preferably from patients exhibiting or prone to an undesirable immune response. For example, antibodies are preferably obtained and derived from allergy patients, and most preferably from allergy patients allergic to the polypeptide of interest. Preferred antibodies for use with the disclosed method are those that are reactive to the polypeptide of interest.

Monoclonal antibodies can be generated using standard techniques. For the present method, the monoclonal antibodies should be selected for reactivity with the polypeptide of interest. Production of antibody fragments that retain binding specificity can also be accomplished using established techniques. Preferred antibody fragments for use in the disclosed method are Fabs.

Antibodies for use in the disclosed method are preferably derived from combinatorial antibody libraries. Techniques for generating combinatorial antibody libraries are known. For example, the method of Barbas et al. (Barbas et al., J. Mol. Biol. 230:812-823 (1993)) can be employed to generate and screen combinatorial phage display immunoglobulin libraries for use in the disclosed method. The following example illustrates how such combinatorial antibody libraries can be generated. Blood samples can be obtained from a patient (or pooled from several patients) manifesting an immune response to a polypeptide of interest. Peripheral blood monocytes can be prepared by centrifugation through a Ficoll-Paque (Pharmacia) density gradient (Steinberger et al., Allergy Clin. Immunol. 96:209-218 (1995)). Standard techniques such as guanadinium HCl extraction can be used to prepare mRNA from these cells (Davis et al., Basic Methods in Molecular Biology (Elsevier, New York, 1986)). Random DNA hexamers or immunoglobulin sequence-specific oligonucleotides can then be used to prime reverse transcription.

Reverse transcribed cDNA can be used as the template in PCR reactions to amplify sequences encoding heavy chain and light chain variable and/or constant regions. The sequences of these primers can be chosen to amplify a specific class of immunoglobulin sequences. For example, to amplify IgE sequences, the primer sequences and conditions described by Steinberger et al., J. Biol. Chem. 271:10967-10972 (1996), can be employed. For IgG sequences, primers as described by Tomlinson et al., J. Mol. Biol. 227:776 (1992), or Barbas et al., J. Mol. Biol. 230:812-823 (1993), can be employed.

The products of the PCR amplification reactions can be digested with appropriate restriction enzymes for subcloning into a suitable expression vector, such as the pComb3 expression vector. The pComb3 phagemid vector allows for combinatorial mixing of heavy and light chain variable sequences to generate a library of constructs encoding recombinant Fab fragments. The library can be transformed in E. coli and phage production can be initiated by rescue with helper phage. Since the pComb3 vector fuses the Fab sequences to the gene III coat protein, Fab proteins are presented on the surfaces of the recombinant phage.

Phage carrying sequences encoding Fabs directed against the polypeptide of interest can be identified by “panning”. For panning, the polypeptide of interest can be immobilized in the wells of ELISA plates or on nitrocellulose. After blocking non-specific binding sites through incubation with a solution such as phosphate buffered saline (PBS) containing 3% bovine serum albumin (BSA), the immobilized polypeptide can be incubated with recombinant phage (approximately 10¹² plaque forming units). Unbound phage can then be removed by washing in PBS or in a Tris buffered saline solution containing detergent, such as 0.05% Tween 20. Bound phage can be eluted with a solution containing 0.1% BSA and 0.1 M glycine HCl, pH 2.2.

After neutralization of the elution solution with 2 M Tris, E. coli can be infected with the eluted phage and cultures of phage-infected bacteria grown. Co-infection with helper phage allows production of filamentous phage, which can again be used in the “panning” assay. Several (3 to 5) rounds of panning, phage elution, and bacterial transformation should be performed, after which individual phagemids can be isolated and their encoded immunoglobulin sequences determined.

To produce soluble recombinant Fab fragments, plasmid encoding immunoglobulin sequences of interest (that is, those that react with the polypeptide of interest) can be excised from the phagemid by restriction digest with enzymes (Spe I and Nhe I can be used in the case of pComb3). The linearized plasmid can be separated from phage sequences by agarose gel electrophoresis, recovered from the agarose by glass milk purification and recircularized according to standard DNA ligation protocols. E. coli transformed with the Fab-encoding plasmid can be isolated by antibiotic selection. Production of the Fab protein can be induced through incubation at 30° C. in the presence of isopropyl-1-thio-β-D-galactopyranoside (IPTG) for several hours. Bacteria can then be collected by centrifugation and the supernatant containing the Fab retained. If necessary, Fab can be affinity purified from the bacterial culture medium. This can be accomplished by passing the medium over a chromatography resin to which recombinant or purified native antigen of interest has been coupled.

As described above, an immunocompetition assay can be performed to determine whether an antibody library includes a complete inventory of the polypeptide-specific antibodies. Briefly, pooled recombinant Fabs can be preincubated with immobilized polypeptide of interest and the immobilized polypeptide can then be incubated with patient serum. The extent to which recombinant Fab prevented binding of serum antibody to polypeptide can be assessed by incubating the immobilized polypeptide with a reporter-coupled secondary antibody specific for the antibody Fc domain. As a control, uncompeted serum antibody binding can be determined using polypeptide which had not been preincubated with Fab or had been incubated with nonsense Fab.

Many other useful techniques for the isolation and production of antibodies, monoclonal antibodies, and antibody fragments are described in Harlow and Lane, “Antibodies-A Laboratory Manual” (Cold Spring Harbor, 1988), “Monoclonal Antibodies” (Kennett et al., eds., Plenum Press, 1980), and Steward and Steensgaard, “Antibody Affinity: Thermodynamic Aspects and Biological Significance” (CRC Press, 1983).

B. Polypeptides

The disclosed method can be used to reduce or eliminate the potential for an undesirable immune response to any protein, peptide, or polypeptide of interest. Preferred targets for selection in the disclosed method include enzymes, therapeutic proteins and peptides, allergens and other antigenic proteins and peptides, and any other protein or peptide intended to be introduced into humans, other mammals, and other animals.

Where an enzyme is used as the polypeptide of interest, it is preferred that the characteristic retained in the mutant peptide be its enzymatic activity. Any enzyme can be used with the disclosed method. Enzymes that are antigenic are preferred and enzymes that are allergenic are most preferred. Peptides and proteins that have a bioactivity are also preferred polypeptides for use with the disclosed method. It is preferred that when a bioactive polypeptide is used, the bioactivity of the polypeptide is the retained characteristic of the mutant polypeptides.

Preferred polypeptides for use in the disclosed method are antigens. Preferred antigens for use with the disclosed method are allergens. Many allergens are known that elicit allergic responses, which may range in severity from mildly irritating to life-threatening. Allergens include polypeptides from insects, foods, molds, dust, pollens, plants, fish, shellfish, and mammals. Any of these can be used with the disclosed method.

1. Mutant Polypeptides

A mutant polypeptide is a polypeptide having an amino acid sequence that differs in at least one position from a polypeptide of interest. Preferably, mutant polypeptides are polypeptides expressed from mutagenized nucleic acid encoding a polypeptide of interest. These mutant polypeptides are the raw material for selection of those mutant polypeptides that elicit less of an undesirable immune response while retaining one or more desired characteristics. By operation of the disclosed method there are several classes of mutant polypeptides. These include mutant polypeptides in general, which include any mutant form of the polypeptide of interest regardless of its potential for eliciting an undesirable immune response or the presence or absence of desired characteristics; mutant polypeptides having reduced potential for eliciting an undesirable immune response, which are mutant polypeptides that exhibit a reduction in measurable immune characteristic(s) in a chosen assay (or suite of assays) relative to the non-mutant polypeptide (that is, the polypeptide of interest); mutant polypeptides that fail to elicit an undesirable immune response, which is a subset of the class of mutant polypeptides having reduced potential to elicit an undesirable immune response that have no detectable measurable immune characteristic(s) in a chosen immune assay (or suite of assays); mutant polypeptides that retain one or more desired characteristics; and mutant polypeptides that have both reduced potential for eliciting an undesirable immune response and that retain one or more desired characteristics, which are the desired result of the disclosed method.

Members of several of these classes of mutant polypeptides are to be identified in the disclosed method. Such mutant polypeptides are referred to as identified mutant polypeptides or by the feature by which the mutant polypeptide is identified. Thus, for example, mutant polypeptides identified in the disclosed method as being less reactive with IgE can be referred to as identified mutant polypeptides having less IgE reactivity or simply as less reactive mutant polypeptides.

The class of mutant polypeptides failing to elicit an undesirable immune response refers to mutant polypeptides having no detectable measurable immune characteristic(s) in a given immune assay or assays, within the limits of detection in the assay(s). Thus, the measurable immune characteristic of a mutant polypeptide need not be below any absolute level to be considered a mutant polypeptide that fails to elicit an undesirable immune response. Furthermore, the immune assay or assays can be freely chosen such that, if, in the assay used as the standard, the mutant polypeptide exhibits no measurable immune characteristic, the mutant polypeptide can be considered to be a mutant polypeptide that fails to elicit an undesirable immune response even if the mutant polypeptide might exhibit a measurable immune characteristic in a different assay.

Mutant polypeptides contain mutations that can be identified by comparison of the mutant polypeptide to the polypeptide of interest. Such mutations are referred to as identified mutations. Many techniques are available for determining differences between a mutant polypeptide and the polypeptide of interest. It is preferred that mutations be identified by comparing the nucleotide sequence encoding the mutant polypeptide and the nucleotide sequence encoding the polypeptide of interest.

In recognition of the relationship between the polypeptide of interest and mutant polypeptides, the polypeptide of interest can be referred to as the non-mutant polypeptide—that is, the form of the polypeptide of interest encoded by the nucleic acid prior to mutagenesis. This does not mean that the non-mutant polypeptide is non-mutant in any absolute sense. Rather, the term is used merely to reflect the relationship between the original polypeptide of interest and the mutant polypeptides produced in the disclosed method. Thus, for example, a “mutant” form of a polypeptide can be used as the polypeptide of interest and, in this context, becomes the “non-mutant” polypeptide.

Mutant polypeptides that exhibit less of an undesirable immune response can be used for any purpose. For example, such mutant polypeptides can be used for a known use of the polypeptide of interest. Such a use is preferably related to the retained characteristic of the mutant polypeptide. Preferred uses for the disclosed mutant polypeptides are uses involving introduction of the mutant polypeptide into an animal body and uses involving exposure of an animal to the mutant polypeptide. Such uses include use in food or nutritional supplements, use as a therapeutic, and use as a treatment. A preferred use for mutant polypeptides is use in immunotherapy or other method for the alteration, manipulation, or modulation of the immune system. Many techniques for these uses are known and can be used with the disclosed mutant polypeptides. For example, numerous techniques for the administration of drugs and therapeutics can be used for the disclose polypeptides. Many compositions, formulations, and devices for drug delivery are known and can be used to administer the disclosed peptides.

Mutant polypeptides identified in the disclosed method, or derived from mutant polypeptides identified in the disclosed method, can also be used in a composition including one or more immunomodulatory molecules or in combination with one or more immunomodulatory molecules. Preferred immunomodulatory molecules include interleukins, such as IL-12, and adjuvant molecules or compositions. Many adjuvants are known and can be used with the disclosed mutant polypeptides. Preferably, the mutant polypeptide and immunomodulatory molecule are physically associated. Such physical association can include, for example, co-encapsulation of the polypeptide and the immunomodulatory molecule, covalent association of the polypeptide and the immunomodulatory molecule, or non-covalent association of the polypeptide and the immunomodulatory molecule. Examples of covalent association include chemical coupling or crosslinking of the mutant polypeptide and immunomodulatory molecule, or, where the immunomodulatory molecule is a polypeptide, a fusion polypeptide of the mutant polypeptide and immunomodulatory polypeptide.

Mutant polypeptides that exhibit less of an undesirable immune response can be expressed in transgenic plants or animals. This is useful, for example, where the polypeptide of interest from which a mutant peptide was derived is naturally present in the plant or animal. Transgenic plants or animals expressing the mutant polypeptides have two purposes. First, they can be used as a source of mutant protein (for use as a therapeutic or in immunotherapy, for example) and second, appropriately modified plants or animals can be substituted for the original plant or animal, thereby reducing the risk of an undesirable immune response when the plant or animal is consumed or when products derived from the plant or animal are introduced into an animal. For example, it is possible that eating such a transgenic animal or plant could have either or both of two effects: (1) not imparting an undesirable immune response on their own and (2) conferring protection from the unmodified source by acting as an immunotherapeutic agent for the unmodified source.

Methods for engineering of plants and animals are well known and have been for a decade. For example, for plants see Day, Crit. Rev. Food Sci. & Nut. 36(S):549-567 (1996), and Fuchs and Astwood, Food Tech. 83-88 (1996). Methods for making recombinant animals are also well established. See, for example, Colman, Biochem. Soc. Symp. 63:141-147 (1998); Espanion and Niemann, DTW Dtxch Tierarztl Wochenschr 103(8-9):320-328 (1996); and Colman, Am. J. Clin. Nutr. 63(4):639S-6455S. One can also induce site specific changes using homologous recombination and/or triplex forming oligomers. See, for example, Rooney and Moore, Proc. Natl. Acad. Sci. USA 92:2141-2149 (1995); Agrawal el al., BioWorld Today, 9(41):1.

Mutant polypeptides identified in the disclosed method, or derived from mutant polypeptides identified in the disclosed method, can be fused to one or more other polypeptides. The other polypeptides, referred to herein as fusion partner polypeptides, can be any peptide or protein and can be fused to the mutant polypeptide for any purpose. It is preferred that the fusion partner polypeptide have immunomodulatory activity. Preferred fusion partner polypeptides include interleukins, such as IL-12, and adjuvant polypeptides. A fusion of an allergen and an interleukin has been shown to be more effective for stimulating allergic desensitization (Kim et al., J. Immunology 158:4137-4144 (1997)).

2. Polypeptide Characteristics

The disclosed method is concerned with reducing undesirable immune responses to polypeptides of interest by identifying mutant forms of the polypeptides with less potential for eliciting an undesirable immune response. Such mutant polypeptides having less potential for eliciting an undesirable immune response are most useful when they retain characteristics of the original polypeptide. The retained characteristic can be any useful or desirable characteristic of a polypeptide, such as enzymatic activity, bioactivity, and T cell activation.

A characteristic is retained when the characteristic is present in the mutant polypeptide. To be considered retained, the characteristic need not be at the same level as in the polypeptide of interest, although this is preferable. In general, the characteristic need be retained only to a useful level. Where the desired characteristic is an immunological characteristic (for example, antibody reactivity, T cell activation, or desensitization activity), such a characteristic can be assessed using techniques described or referred to elsewhere herein. In general, an increase in the level of the desired characteristic is also desirable and is not intended to be excluded from the disclosed method.

Bioactivity is another preferred characteristic to be retained by mutant polypeptides. A bioactivity can be any biological effect or function that a peptide or protein may have. For example, bioactivities include specific binding to biomolecules (for example, receptor ligands), hormonal activity, cytokine activity, and inhibition of biological activity or interactions of other biomolecules (for example, agonists and antagonists of receptor binding), enzymatic activity, anticancer activity, immunosuppressive activity, immunostimulatory activity, immune characteristic, alteration of the function of immune system cells, antibiotic activity, antiviral activity, and trophic activity. Bioactivity can be measured and detected using appropriate techniques and assays known in the art. Antibody reactivity and T cell activation can be considered bioactivities. In view of this, desired characteristics involving bioactivity can be classified herein as bioactivity in general, bioactivity other than antibody reactivity and T cell activation, antibody reactivity and T cell activation, antibody reactivity, and T cell activation. Bioactivity can also be assessed in vivo where appropriate. This can be the most accurate assessment of the presence of a useful level of the bioactivity of interest. Enzymatic activity can be measured and detected using appropriate techniques and assays known in the art.

In the case of viral proteins—for use with, for example, viral vectors, therapeutic viruses, and viral capsid delivery compositions—desired characteristics to be retained can include the ability to assemble into a viral particle or capsid and the ability to infect or enter cells. Such characteristics are useful where the delivery properties of the viral proteins are of interest.

C. Nucleic Acid Encoding Target Polypeptides

The disclosed method preferably involves selection of mutant forms of a protein or peptide of interest. These mutant forms are produced by mutagenizing nucleic acid molecules encoding the polypeptide and expressing the mutant polypeptides from the mutated nucleic acid molecules. Thus, the disclosed method requires nucleic acid molecules encoding the polypeptide of interest be available or obtainable. Many nucleic acids encoding polypeptides are known and others can be obtained using well established cloning techniques.

The disclosed method preferably involves expression of the nucleic acid encoding the polypeptide of interest, and mutant forms of this nucleic acid, to produce the polypeptide that it encodes. The nucleic acid encoding the polypeptide of interest can be expressed using any suitable expression sequences. Numerous expression sequences are known and can be used for expression of the gene of interest. The nucleic acid encoding the polypeptide of interest may be expressed not only directly, but also as a fusion with another polypeptide, preferably a reporter protein that can be used to identify and exclude mutagenized nucleic acids having gross mutations such as frameshifts and large deletions as described above.

The nucleic acid encoding the polypeptide of interest can encode a fusion protein including sequence encoding the polypeptide of interest and, downstream of this sequence, sequence encoding a reporter protein. This arrangement makes expression of the reporter protein dependent on expression of the polypeptide of interest. That is, if the nucleic acid encoding the polypeptide of interest has a gross mutation such as a frameshift or large deletion, this will affect the reporter protein and loss of expression of the reporter protein can serve as a convenient sign that such a mutation is present. Gross mutations are undesirable since they are likely to have reduced potential for eliciting an undesirable immune response but not retain the desired characteristic(s).

The reporter protein can be any protein or peptide the expression of which can be detected. Preferred polypeptides for use as a reporter protein are polypeptides required for cell growth. By using a polypeptide necessary for cell growth, elimination of expression by gross mutations will cause death of those cells. In this way, cells that harbor undesirable gross mutations will not be present in the pool of cells expressing mutant polypeptides.

The reporter protein can also be any polypeptide the expression of which can be detected either directly or indirectly. These include enzymes, such as β-galactosidase, luciferase, and alkaline phosphatase, that can produce specific detectable products, and polypeptides that can be directly detected. Virtually any polypeptide can be directly detected by using, for example, specific antibodies to the polypeptide. A preferred reporter protein that can be directly detected is the green fluorescent protein (GFP). GFP, from the jellyfish Aequorea victoria, produces fluorescence upon exposure to ultraviolet light without the addition of a substrate (Chalfie et al., Science 263:802-5 (1994)). A number of modified GFPs have been created that generate as much as 50-fold greater fluorescence than does wild type GFP under standard conditions (Cormack et al., Gene 173:33-8 (1996); Zolotukhin et al., J. Virol 70:4646-54 (1996)). This level of fluorescence allows the detection of low levels of expression in cells.

Reporter proteins producing a fluorescent signal are useful since such a signal allows cells to be sorted using FACS. Another way of sorting cells based on expression of the reporter protein involves using the reporter protein as a hook to bind cells. For example, a cell surface protein such as a receptor protein can be bound by a specific antibody. Cells expressing such a reporter protein can be captured by, for example, using antibodies bound to a solid substrate, using antibodies bound to magnetic beads, or capturing antibodies bound to the reporter protein. Many techniques for the use of antibodies as capture agents are known and can be used with the disclosed method.

The reporter protein can also be a polypeptide that regulates the expression of another gene. This allows detection of expression of the reporter protein by detecting expression of the regulated gene. For example, a repressor protein can be used as the reporter protein. Inhibition of expression of the reporter protein would then result in derepression of the regulated gene. This type of indirect detection allows positive detection of inhibition of the expression of the reporter protein by the affector RNA molecule. One preferred form of this type of regulation is the use of an antibiotic resistance gene regulated by a repressor protein used as the reporter protein. By exposing the host cells to the antibiotic, only those cells in which expression of the reporter gene has been inhibited will grow since expression of the antibiotic resistance gene will be derepressed.

It is preferred that the reporter be chosen to insure that it does not interfere with the bioactivity of the polypeptide of interest or be coupled to the polypeptide of interest such that it can easily be removed (prior to assessment of bioactivity) by standard methods such as protease cleavage.

It is understood that the disclosed invention is not limited to the particular methodology, protocols, and reagents described herein as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

Those skilled in the art will recognize, or be able to ascertain using no more than routine experimentation, many equivalents to the specific embodiments of the invention described herein. Such equivalents are intended to be encompassed by the following claims.

I claim:

1. A method comprising
providing a collection of mutant polypeptides wherein the amino acid sequence of each mutant polypeptide differs in at least one position from a polypeptide of interest, and
identifying those mutant polypeptides within the collection that (1) have an alteration in antibody reactivity compared to the polypeptide of interest, and (2) retain at least one desired characteristic,
wherein alteration in the antibody reactivity is determined by exposing the mutant polypeptides to individual antibodies or antibody fragments that are monospecific for the polypeptide of interest.

2. The method of claim 1 wherein the collection of mutant polypeptides is provided by
mutagenizing nucleic acid encoding a polypeptide of interest, and
expressing the mutagenized nucleic acid to produce the collection of mutant polypeptides.

3. The method of claim 2 wherein the nucleic acid encoding the polypeptide of interest is mutagenized such that a collection of randomly mutagenized nucleic acids is produced which encodes a collection of randomly mutant polypeptides.

4. The method of claim 1 wherein either or both the antibody reactivity and the alteration in the antibody reactivity are associated with an undesirable immune response.

5. The method of claim 2 wherein the antibody reactivity is the undesirable immune response, wherein the undesirable immune response is mediated by the antibody reactivity, wherein the antibody reactivity is involved in the undesirable immune response, wherein the antibody reactivity is associated with the undesirable immune response, or a combination of these.

6. The method of claim 1 wherein the antibodies or antibody fragments that are monospecific for the polypeptide are either monoclonal antibodies derived from mammals or antibodies or antibody fragments derived from a combinatorial library.

7. The method of claim 6 wherein the antibodies or antibody fragments are antibody fragments derived from a combinatorial library.

8. The method of claim 1 wherein the antibody reactivity is reactivity to IgA antibodies, reactivity to IgD antibodies, reactivity to IgE antibodies, reactivity to IgG antibodies, or reactivity to IgM antibodies.

9. The method of claim 8 wherein the antibody reactivity is reactivity to IgE antibodies that are reactive to the polypeptide of interest.

10. The method of claim 9 wherein the desired characteristic is T cell activation.

11. The method of claim 9 wherein the desired characteristic is an immune characteristic involved in desensitization.

12. The method of claim 1 wherein identification of mutant polypeptides that have an alteration in antibody reactivity is carried out prior to, simultaneous with, or following identification of mutant polypeptides that retain the desired characteristic.

13. The method of claim 1 wherein the desired characteristic is a bioactivity present in the polypeptide of interest.

14. The method of claim 13 wherein the bioactivity is selected from the group consisting of enzymatic activity, receptor binding, anticancer activity, immunosuppressive activity, immunostimulatory activity, immune characteristic, alteration of the function of immune system cells, antibiotic activity, antiviral activity, and trophic activity.

15. The method of claim 14 wherein the bioactivity is T cell activation or B cell activation.

16. The method of claim 14 wherein the bioactivity is an immune characteristic involved in desensitization.

17. The method of claim 14 wherein the bioactivity is an alteration of the function of immune system cells, wherein the cells are dendritic cells, macrophages, mast cells, basophils, or eosinophils.

18. The method of claim 13 wherein the polypeptide of interest is a viral protein and wherein the bioactivity is mediation of viral assembly and infectivity or cell entry.

19. The method of claim 1 further comprising
identifying the mutations present in the identified mutant polypeptides, and
combining two or more of the identified mutations in a single mutant polypeptide.

20. The method of claim 1 further comprising expressing the polypeptide in a transgenic animal or plant.

21. The method of claim 20 wherein the polypeptide of interest naturally occurs in non-transgenic animals or plants of the same type as the transgenic animal or plant.

22. The method of claim 1 further comprising
administering one or more times to an individual one or more polypeptides each derived from at least one of the identified mutant polypeptides.

23. The method of claim 22 wherein each of the one or more polypeptides is one of the identified mutant polypeptides or a polypeptide combining mutations from two or more of the identified mutant polypeptides.

24. The method of claim 22 wherein the polypeptide is administered in combination with an immunomodulatory molecule.

25. The method of claim 24 wherein the polypeptide and immunomodulatory molecule are physically associated.

26. The method of claim 25 wherein the polypeptide and immunomodulatory molecule are co-encapsulated, covalently associated, or non-covalently associated.

27. The method of claim 26 wherein the polypeptide and immunomodulatory molecule are chemically coupled.

28. The method of claim 24 wherein the immunomodulatory molecule is a polypeptide fused to the polypeptide.

29. A polypeptide derived from at least one mutant polypeptide identified by the method of claim 1, wherein the polypeptide (1) has an alteration in antibody reactivity compared to the polypeptide of interest, and (2) retains the desired characteristic.

30. The polypeptide of claim 29 wherein the polypeptide is one of the identified mutant polypeptides or a polypeptide combining mutations from two or more of the identified mutant polypeptides.

31. The polypeptide of claim 29 wherein the mutant polypeptide has an alteration in at least one measurable immune characteristic associated with the undesirable immune response,
wherein the measurable immune characteristic is reactivity to IgE antibodies that are reactive to the polypeptide of interest.

32. The polypeptide of claim 29 wherein the polypeptide is produced in a transgenic plant or animal.

33. A fusion protein comprising the polypeptide of claim 29 and a polypeptide that has immunomodulatory activity.

34. A composition comprising the polypeptide of claim 29 and an immunomodulatory molecule.

35. The composition of claim 34 wherein the polypeptide and immunomodulatory molecule are physically associated.

36. The composition of claim 35 wherein the polypeptide and immunomodulatory molecule are co-encapsulated, covalently associated, or non-covalently associated.

37. The composition of claim 36 wherein the polypeptide and immunomodulatory molecule are chemically coupled.

38. The composition of claim 34 wherein the immunomodulatory molecule is a polypeptide fused to the polypeptide.

39. A nucleic acid encoding the polypeptide of claim 29.

40. The nucleic acid of claim 39 wherein the nucleic acid comprises a vector for expression of the polypeptide in a recombinant host.

41. The nucleic acid of claim 40 wherein the polypeptide is expressed in a transgenic plant or animal.

42. A transgenic plant expressing the polypeptide of claim 29.

43. The transgenic plant of claim 42 wherein the polypeptide of interest naturally occurs in non-transgenic plants of the same type as the transgenic plant.

44. A transgenic animal expressing the polypeptide of claim 29.

45. The transgenic animal of claim 44 wherein the polypeptide of interest naturally occurs in non-transgenic animals of the same type as the transgenic animal.

46. A method comprising
providing a collection of mutant polypeptides wherein the amino acid sequence of each mutant polypeptide differs in at least one position from a polypeptide of interest, wherein the polypeptide of interest is an allergen, and
identifying those mutant polypeptides within the collection that (1) exhibit less of, or have less potential to exhibit, an allergic response than the polypeptide of interest, and (2) retain at least one desired characteristic.

47. The method of claim 46 wherein the collection of mutant polypeptides is provided by
mutagenizing nucleic acid encoding a polypeptide of interest, and
expressing the mutagenized nucleic acid to produce the collection of mutant polypeptides.

48. The method of claim 46 wherein identification of mutant polypeptides that exhibit less of, or have less potential to exhibit, an allergic response is accomplished by exposing the mutant polypeptides to individual IgE antibodies or antibody fragments that are reactive to the polypeptide of interest.

49. The method of claim 48 wherein the IgE antibodies or antibody fragments that are reactive to the polypeptide of interest are either monoclonal IgE antibodies derived from mammals or IgE antibodies or antibody fragments derived from a combinatorial IgE library.

50. The method of claim 49 wherein the IgE antibodies or antibody fragments are IgE antibody fragments derived from a combinatorial IgE library.

51. The method of claim 46 wherein the desired characteristic is T cell activation.

52. The method of claim 46 wherein the desired characteristic is an immune characteristic involved in allergic desensitization.

53. The method of claim 46 wherein identification of mutant polypeptides that exhibit less of, or have less potential to exhibit, an allergic response is carried out prior to, simultaneous with, or following identification of mutant polypeptides that retain the desired characteristic.

54. A method to treat an individual to reduce the allergic response to an allergen, the method comprising
administering one or more times to the individual one or more polypeptides each derived from at least one mutant polypeptide identified by the method of claim 46.

55. The method of claim 54 wherein each of the one or more polypeptides is one of the identified mutant polypeptides or a polypeptide combining mutations from two or more of the identified mutant polypeptides.

56. The method of claim 54 wherein the polypeptide is administered in combination with an immunomodulatory molecule.

57. The method of claim 56 wherein the polypeptide and immunomodulatory molecule are physically associated.

58. The method of claim 57 wherein the polypeptide and immunomodulatory molecule are co-encapsulated, covalently associated, or non-covalently associated.

59. The method of claim 58 wherein the polypeptide and immunomodulatory molecule are chemically coupled.

60. The method of claim 56 wherein the immunomodulatory molecule is a polypeptide fused to the polypeptide.

61. A method comprising
providing a collection of mutant polypeptides wherein the amino acid sequence of each mutant polypeptide differs in at least one position from a polypeptide of interest,
wherein each mutant polypeptide is part of a fusion polypeptide comprising the mutant polypeptide and a reporter protein, and
identifying those mutant polypeptides within the collection that (1) exhibit less of, or have less potential to exhibit, at least one undesirable immune response than the polypeptide of interest, and (2) retain at least one desired characteristic, and identifying fusion proteins with a functional reporter protein.

62. The method of claim 61 wherein the collection of mutant polypeptides is provided by
mutagenizing nucleic acid encoding a polypeptide of interest, and
expressing the mutagenized nucleic acid to produce the collection of mutant polypeptides,
wherein the mutagenized nucleic acid is operably linked to nucleic acid encoding the reporter protein such that the linked nucleic acids encode the fusion polypeptide comprising a mutant polypeptide and the reporter protein.

63. The method of claim 61 wherein identification of mutant polypeptides that exhibit less of, or have less potential to exhibit, the undesirable immune response is accomplished by identifying an alteration in at least one measurable immune characteristic associated with the undesirable immune response,
wherein either or both of the measurable immune characteristic and the alteration in the measurable immune characteristic are associated with the undesirable immune response.

64. The method of claim 63 wherein the measurable immune characteristic is antibody reactivity.

65. The method of claim 64 wherein the alteration in the measurable immune characteristic is a reduction in antibody reactivity.

66. The method of claim 64 wherein alteration in the measurable immune characteristic is determined by exposing the mutant polypeptides to individual antibodies or antibody fragments that are reactive to the polypeptide of interest,
wherein the antibodies or antibody fragments that are reactive to the polypeptide are either monoclonal antibodies derived from mammals or antibodies or antibody fragments derived from a combinatorial library.

67. The method of claim 66 wherein the antibodies or antibody fragments are antibody fragments derived from a combinatorial library.

68. The method of claim 64 wherein the measurable immune characteristic is reactivity to IgA antibodies, reactivity to IgD antibodies, reactivity to IgE antibodies, reactivity to IgG antibodies, or reactivity to IgM antibodies.

69. The method of claim 68 wherein the measurable immune characteristic is reactivity to IgE antibodies that are reactive to the polypeptide of interest.

70. The method of claim 69 wherein alteration in the measurable immune characteristic is determined by exposing the mutant polypeptides to individual IgE antibodies or antibody fragments that are reactive to the polypeptide of interest,
wherein the IgE antibodies or antibody fragments that are reactive to the polypeptide of interest are either monoclonal IgE antibodies derived from mammals or IgE antibodies or antibody fragments derived from a combinatorial IgE library.

71. The method of claim 70 wherein the IgE antibodies or antibody fragments are IgE antibody fragments derived from a combinatorial IgE library.

72. The method of claim 69 wherein the desired characteristic is T cell activation.

73. The method of claim 69 wherein the desired characteristic is an immune characteristic involved in desensitization.

74. The method of claim 63 wherein the measurable immune characteristic is T cell activation, B cell activation, NK cell activation, or alteration of the function of dendritic cells, macrophages, mast cells, basophils, or eosinophils.

75. The method of claim 63 wherein the measurable immune characteristic is the undesirable immune response, wherein the undesirable immune response is mediated by the measurable immune characteristic, wherein the measurable immune characteristic is involved in the undesirable immune response, wherein the measurable immune characteristic is associated with the undesirable immune response, or a combination of these.

76. The method of claim 61 wherein identification of mutant polypeptides that exhibit less of, or have less potential to exhibit, the undesirable immune response is carried out prior to, simultaneous with, or following identification of mutant polypeptides that retain the desired characteristic.

77. The method of claim 61 wherein the undesirable immune response is reactivity to IgA antibodies, reactivity to IgD antibodies, reactivity to IgE antibodies, reactivity to IgG antibodies, reactivity to IgM antibodies, B cell activation, T cell activation, NK cell activation, or a combination.

78. The method of claim 61 wherein the undesirable immune response is humoral immune response, cellular immune response, or allergic response.

79. A method comprising
providing a collection of mutant polypeptides wherein the amino acid sequence of each mutant polypeptide differs in at least one position from a polypeptide of interest, and
identifying those mutant polypeptides within the collection that (1) exhibit less of, or have less potential to exhibit, at least one undesirable immune response than the polypeptide of interest, and (2) retain at least one desired characteristic, and identifying fusion proteins with a functional reporter protein,
wherein identification of mutant polypeptides that exhibit less of, or have less potential to exhibit, the undesirable immune response is accomplished by identifying an alteration in a measurable immune characteristic other than antibody reactivity that is associated with the undesirable immune response.

80. The method of claim 79 wherein the collection of mutant polypeptides is provided by
mutagenizing nucleic acid encoding a polypeptide of interest, and
expressing the mutagenized nucleic acid to produce the collection of mutant polypeptides.

81. The method of claim 80 wherein the nucleic acid encoding the polypeptide of interest is mutagenized such that a collection of randomly mutagenized nucleic acids is produced which encodes a collection of randomly mutant polypeptides.

82. The method of claim 79 wherein either or both of the measurable immune characteristic and the alteration in the measurable immune characteristic are associated with the undesirable immune response.

83. The method of claim 79 wherein the measurable immune characteristic is T cell activation, B cell activation, NK cell activation, or alteration of the function of dendritic cells, macrophages, mast cells, basophils, or eosinophils.

84. The method of claim 79 wherein the undesirable immune response is a humoral immune response, a cellular immune response, or an allergic response.

85. The method of claim 79 wherein identification of mutant polypeptides that exhibit less of, or have less potential to exhibit, the undesirable immune response is carried out prior to, simultaneous with, or following identification of mutant polypeptides that retain the desired characteristic.

86. The method of claim 79 wherein the desired characteristic is a bioactivity present in the polypeptide of interest.

87. The method of claim 86 wherein the bioactivity is selected from the group consisting of enzymatic activity, receptor binding, anticancer activity, immunosuppressive activity, immunostimulatory activity, immune characteristic, alteration of the function of immune system cells, antibiotic activity, antiviral activity, and trophic activity.

88. The method of claim 79 further comprising
identifying the mutations present in the identified mutant polypeptides, and
combining two or more of the identified mutations in a single mutant polypeptide.
